(19) United States
(12) Patent Application Publication
(10) Pub. No.: US 2026/0187379 A1
(43) Pub. Date: Jul. 2, 2026

(54) METHOD AND APPARATUS WITH EVENT EXTRACTION

(71) Applicant: Samsung Electronics Co., Ltd., Suwon-si (KR)

(72) Inventors: Inkyu CHOI, Suwon-si (KR); Jehun JEON, Suwon-si (KR); Woojin JEONG, Suwon-si (KR); Jiseung JEONG, Suwon-si (KR); GyuBum HAN, Suwon-si (KR)

(73) Assignee: Samsung Electronics Co., Ltd., Suwon-si (KR)

(21) Appl. No.: 19/274,902

(22) Filed: Jul. 21, 2025

(30) Foreign Application Priority Data
Dec. 26, 2024 (KR) 10-2024-0197386

(51) Int. Cl. G06F 40/40; G06F 40/279

(52) U.S. Cl. CPC G06F 40/40; G06F 40/279

(57) ABSTRACT

A processor-implemented method including generating input data based on raw text and a prompt for a predetermined domain, generating, through a large language model (LLM), event candidates corresponding to the raw text, verifying the event candidates based on structured knowledge as target events, normalizing the target events using information related to the predetermined domain, and extracting the normalized target events.

### CROSS-REFERENCE TO RELATED APPLICATIONS

This application claims the benefit under 35 USC § 119(a) of Korean Patent Application No. 10-2024-0197386, filed on Dec. 26, 2024, in the Korean Intellectual Property Office, the entire disclosure of which is incorporated herein by reference for all purposes.

### BACKGROUND

### 1. Field

The following description relates to method and apparatus with event extraction.

### 2. Description of Related Art

In a neural network model such as a large language model (LLM), an “event” may primarily refer to a significant occurrence, activity, or state change that occurs while the model performs a predetermined task or predicts and/or generates a response or an answer. For example, an event may occur when a user inputs a question or gives a command, when the neural network model receives, understands, and processes the question or command input by the user, when the neural network model generates a prediction or an answer based on the input data, and/or when the neural network model generates a final result and provides a response to the user. Events play an important role in the operation of a neural network model, and each event may affect how the neural network model responds and learns.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a general aspect, here is provided a processor-implemented method including generating input data based on raw text and a prompt for a predetermined domain, generating, through a large language model (LLM), event candidates corresponding to the raw text, verifying the event candidates based on structured knowledge as target events, normalizing the target events using information related to the predetermined domain, and extracting the normalized target events.

The prompt may be predefined for respective items of a predefined event to extract the respective items and the predefined event may include a syntax having any one or any combination of trigger words corresponding to the predetermined domain, an event type corresponding to the event, a role related to the event, and an attribute value corresponding to the event.

The generating of the event candidates may include obtaining, through the LLM, components of a detected event from the raw text and generating the event candidates based on the components of the detected event.

The obtaining of the components of the event may include extracting, through the LLM, trigger words corresponding to the detected event and an event type corresponding to the event, extracting, through the LLM, a role related to the detected event by the event type based on the trigger words and the event type, and extracting, through the LLM, an attribute value corresponding to the detected event, based on the trigger words, the event type, and the role.

The obtaining of the components of the event may include determining an extraction order of the components of the event based on a priority of the components of the detected event and obtaining the components of the event according to the extraction order.

The structured knowledge may include one or more of a knowledge subgraph of a domain related to the predetermined domain and the extracted normalized target event.

The verifying of the event candidates may include retrieving structured partial knowledge related to the event candidates from the structured knowledge, obtaining additional information about an event associated with the event candidates based on the structured partial knowledge, and verifying a validity of the event candidates based on the additional information.

The obtaining of the additional information may include extracting, from the structured partial knowledge, a corresponding entity and a knowledge subgraph connected to the corresponding entity, based on roles of the event candidates and obtaining additional information about an event associated with the event candidates using the knowledge subgraph.

The obtaining of the additional information about the event associated with the event candidates using the knowledge subgraph may include obtaining, using dates corresponding to the event candidates and the roles of the event candidates in the knowledge subgraph, the additional information in a list format about associated events that overlap in period and target with the event candidates.

The verifying of the validity of the event candidates based on the additional information may include converting the additional information in a list format into a text format and verifying the validity of the event candidates by applying the additional information converted into a text format, the raw text, and the event candidates to the LLM.

The normalizing of the target events may include normalizing the target events by matching the verified target events to an entity dictionary including information about the predetermined domain.

The normalizing of the target events may include normalizing the target events by standardizing one or more of roles corresponding to the target events and dates corresponding to the target events.

The normalizing of the target events may include normalizing the target events based on the matching entity responsive to a partially matching entity partially matching the roles corresponding to the target events, being in an entity dictionary.

The normalizing of the target events based on the partially matching entity may include forming a trie with a full synonym list of each entity dictionary of a plurality of dictionaries, retrieving a character pattern from within the trie, the retrieved character pattern matching roles of the target events, and normalizing the target events by storing roles in a normalized form and entities matching the roles based on the retrieved character pattern.

The normalizing of the target events may include in response to dates of an entity matching target event dates corresponding to the target events in an entity dictionary, parsing the matching entity based on a rule and normalizing the target events by converting the parsed entity into a date and a period based on a date on which the raw text is written.

The method may include storing the normalized target events in a database.

In a general aspect, here is provided a non-transitory, computer-readable storage medium storing instructions that, when executed by the one or more processors, configure the one or more processors to generate input data based on raw text and a prompt for a predetermined domain, generate, through a large language model (LLM), event candidates corresponding to the raw text, verify the event candidates based on structured knowledge as target events, normalize the target events using information related to the predetermined domain, and extract the normalized target events.

In a general aspect, here is provided an electronic device including processors configured to execute instructions, a memory storing the instructions, and an execution of the instructions configures the processors to generate input data based on raw text and a prompt for a predetermined domain, generate, through a large language model (LLM), event candidates corresponding to the raw text, verify the event candidates based on structured knowledge as target events, normalize the target events using information related to the predetermined domain, and extract the normalized target events.

The prompt may be predefined for respective items of a predefined event to extract the respective items and the event may include a syntax including any one or any combination of trigger words corresponding to the predetermined domain, an event type corresponding to the predefined event, a role related to the predefined event, and an attribute value corresponding to the predefined event.

The processor may be further configured to extract, through the LLM, trigger words corresponding to a detected event from the raw type and an event type corresponding to the detected event, extract, through the LLM, a role related to the detected event by the event type based on the trigger words and the event type, extract, through the LLM, an attribute value corresponding to the detected event, based on the trigger words, the event type, and the role, and generate the event candidates based on components of the detected event including any one or any combination of the trigger words, the event type, the role, and the attribute value.

Other features and aspects will be apparent from the following detailed description, the drawings, and the claims.

### BRIEF DESCRIPTION OF THE DRAWINGS

FIG. 1 illustrates an example method with a knowledge retriever according to one or more embodiments.

FIG. 2 illustrates an example method according to one or more embodiments.

FIG. 3 illustrates an example of raw text and an event according to one or more embodiments.

FIG. 4 illustrates an example of input data according to one or more embodiments.

FIG. 5 illustrates an example method of event candidate generation with a large language model (LLM) according to one or more embodiments.

FIG. 6 illustrates an example method according to one or more embodiments.

FIG. 7 illustrates an example process of retrieving a structured partial knowledge graph (KG) using a knowledge retriever according to one or more embodiments.

FIG. 8 illustrates an example method according to one or more embodiments.

FIG. 9 illustrates an example operation to partially match roles corresponding to target events in a normalization process according to one or more embodiments.

FIG. 10 illustrates an example electronic device according to one or more embodiments.

Throughout the drawings and the detailed description, unless otherwise described or provided, the same or like drawing reference numerals may be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein. The use of the term “may” herein with respect to an example or embodiment, e.g., as to what an example or embodiment may include or implement, means that at least one example or embodiment exists where such a feature is included or implemented, while all examples are not limited thereto.

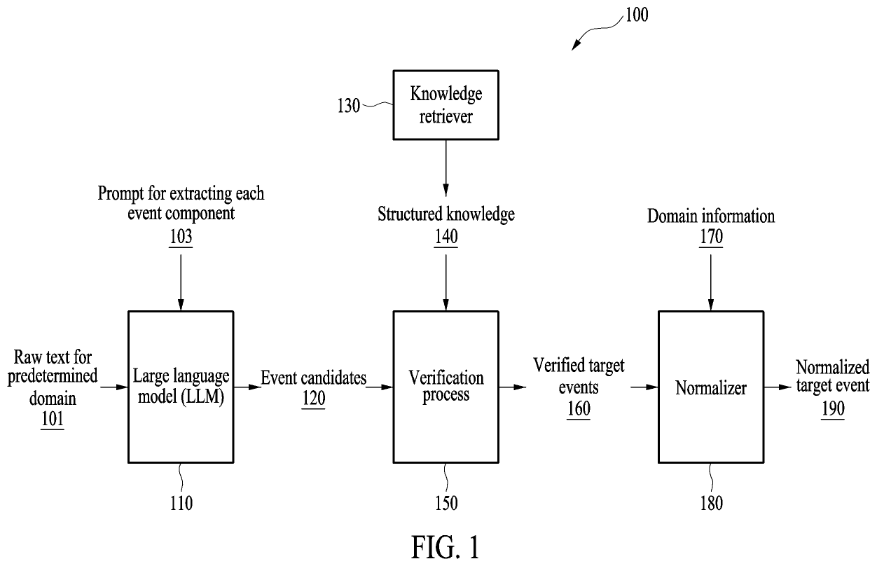

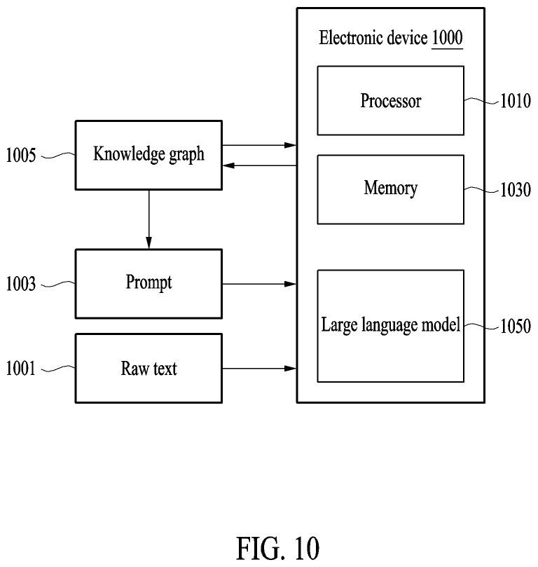

FIG. 1 illustrates an example method with a knowledge retriever according to one or more embodiments. Referring to FIG. 1, in a non-limiting example, a diagram 100 illustrates a method by which an electronic device (e.g., electronic device 1000 of FIG. 10) extracts an event using structured knowledge 140 and domain information 170 retrieved by a knowledge retriever 130 based on a large language model (LLM) 110.

In order to extract a reliable event from a predetermined domain, the electronic device may extract each item of the event by the LLM 110 and a prompt 103 by prompt engineering to generate event candidates 120. Here, prompt engineering (or engineering a prompt) may correspond to a scheme of designing a prompt (i.e., an extraction prompt 103) for the LLM 110 to recognize and extract a predetermined event. For example, prompt engineering may guide the LLM 110 to identify and extract an event through a command such as “extract an important event from the following text.”

The electronic device may verify the event candidates 120 by the structured knowledge 140 and normalize verified target events 160 by utilizing verified domain information 170.

As described above, an “event” may be a significant incident, activity, or state change that occurs during the process of a neural network model (e.g., the LLM 110) as it performs a predetermined task or predicting and/or generating a response or an answer. An event may occur, for example, when a user inputs a question or gives a command, in a process in which a neural network model receives a question or a command from the user where the neural network model then understands and processes the question or the command, and a process in which the neural network model generates a prediction or an answer based on the input data, and/or a process in which the neural network model generates a final result and provides a response to the user. For example, an event may play a role in the operation of a neural network model, and each event may affect how the neural network model reacts and learns.

In an example, the electronic device may extract an event by combining rule-based normalization techniques by the LLM 110 and a normalizer 180 to generate a practically usable event database (DB) from raw text 101 for a predetermined domain. The raw text 101 may be, for example, a phrase including a plurality of words, a paragraph including a plurality of sentences, a document including a plurality of paragraphs, or a context. However, examples are not limited thereto.

The electronic device may extract the event candidates 120 from the raw text 101 based on the LLM 110. The electronic device may extract the event candidates 120 by extracting event components from the raw text 101 for a predetermined domain through the LLM 110. In this case, the electronic device may generate input data based on the raw text 101 for the predetermined domain and the extraction prompt 103 which may extract each event component. The electronic device may generate (or extract) the event candidates 120 corresponding to the raw text 101 by inputting the input data in the LLM 110.

In a process of inputting the raw text 101 in the LLM 110 and extracting the event candidates 120, a process of extracting components of each event may be performed. The electronic device may sequentially extract event components by the LLM 110 to generate the event candidates 120.

The electronic device may have the extraction prompt 103, which is actually input in the LLM 110 for each component of each event. The extraction prompt 103 may include the raw text 101, along with a description of a component desired to be extracted and extraction instructions. Additionally, previously extracted components of an event may be included in the extraction instructions of a subsequent item of an entity. An entity may be an information entity existing in a sentence and may have various meanings depending on the context. For example, the entity “apple” may be the fruit “apple” or the corporation “Apple”, depending on its usage.

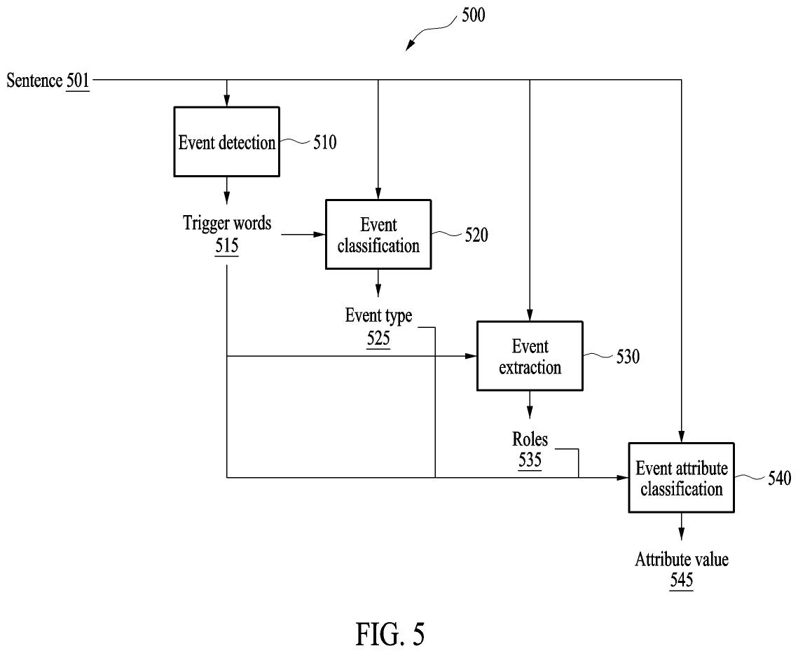

The electronic device may extract all components included in an event, reconstruct the components in the form of an initially defined event, and define the reconstructed components as candidate events. An example of components included in an event is described in greater detail below with reference to FIG. 5.

In an example, the electronic device may verify the event candidates 120 using the structured knowledge 140 in a verification process 150. In this case, the structured knowledge 140 may be obtained by the knowledge retriever 130. The electronic device may verify the event candidates 120 by utilizing the structured knowledge 140 of a corresponding domain (e.g., the predetermined domain to which the raw text 101 belongs). The electronic device may retrieve the structured knowledge 140 associated with the event candidates 120 by the knowledge retriever 130 and use the retrieved structured knowledge 140 to allow the LLM 110 to verify the event candidates 120. Alternatively, the electronic device may verify the event candidates 120 by the LLM 110 using normalized knowledge associated with the event candidates 120 as additional information.

The electronic device may output a normalized target event 190 by normalizing the verified target events 160 based on the domain information 170 using the normalizer 180. The electronic device may store the normalized target event 190 in a database.

The electronic device may perform matching by utilizing an entity dictionary including the predetermined domain information 170 for the verified target events 160. The electronic device may perform matching between the verified target events 160 and entities included in the entity dictionary. The electronic device may normalize, for example, extracted event roles, as illustrated below in FIG. 5, by utilizing entity names and synonyms included in the entity dictionary.

When it is difficult to achieve an exact match between the verified target events 160 and entities included in the entity dictionary, the electronic device may perform partial matching to minimize information loss. Additionally, the electronic device may normalize the verified target events 160 and store the verified target events 160 in a database through a process of mapping relatively expressed dates to accurate times.

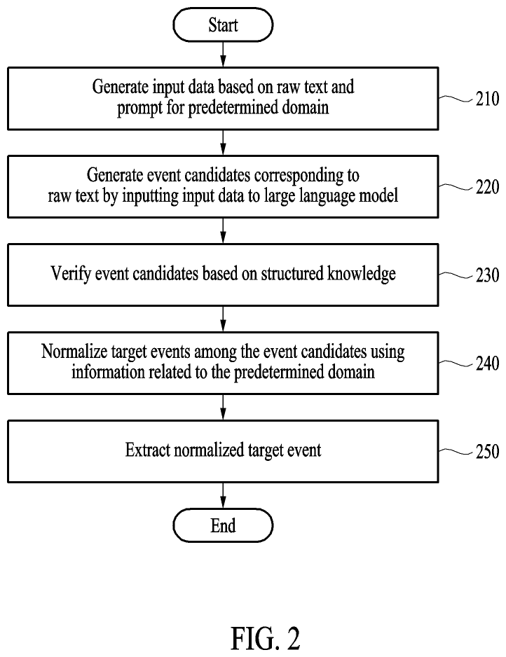

FIG. 2 illustrates an example method according to one or more embodiments. Operations to be described with reference to FIG. 2 and below may be performed sequentially but not necessarily. For example, the order of the operations may change and at least two of the operations may be performed in parallel, or one operation may be performed separately.

Referring to FIG. 2, in a non-limiting example, an electronic device may extract an event through operations 210 to 250. The electronic device may be, for example, an electronic device 1000 illustrated in FIG. 10, but examples are not limited thereto.

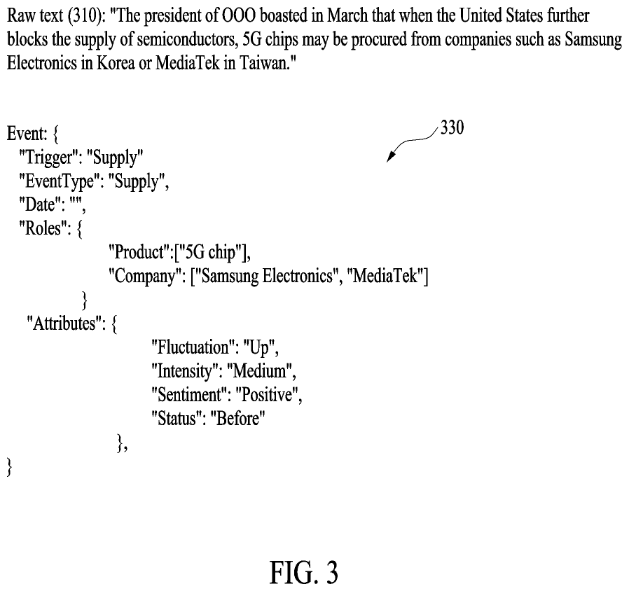

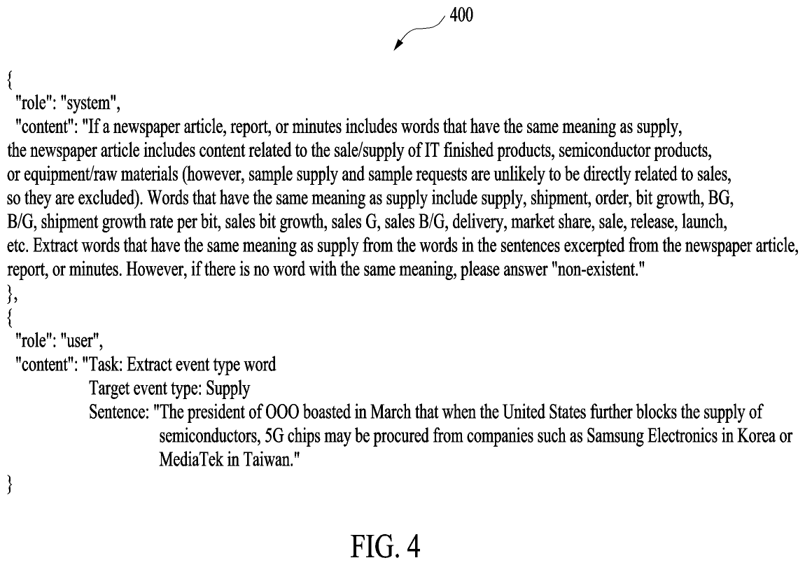

In an example, in operation 210, the electronic device may generate input data based on raw text and a prompt for a predetermined domain. The prompt may be predefined for respective items of an event to extract the respective items. The event may be predefined. The event may include a syntax that includes at least one of, for example, trigger words corresponding to the predetermined domain, an event type corresponding to the event, a role related to the event, and an attribute value corresponding to the event. An example of the raw text and the event is described in greater detail below with reference to FIG. 3. Additionally, an example of input data generated based on the raw text and the prompt is described in greater detail below with reference to FIG. 4.

In an example, in operation 220, the electronic device may generate event candidates corresponding to the raw text by inputting the input data generated in operation 210 in an LLM. The electronic device may receive a response corresponding to input data from the LLM. The electronic device may obtain components of the event based on the response. The electronic device may determine the extraction order of the components of the event based on the priority of the components of the event. Component priorities may vary depending on the event definition. The component priorities may be in the order of event trigger words>event type>event role>event attribute. However, examples are not limited thereto.

When a response corresponds to a component priority, the electronic device may preferentially extract the component of the event from that response. The electronic device may obtain the components of the event from the response in the extraction order.

The electronic device may extract, from the response, trigger words that may correspond to an event and an event type corresponding to that event, for example. The electronic device may extract, from the response, a role related to the event by event type based on the trigger words and the event type. The electronic device may extract, from the response, an attribute value corresponding to the event based on the trigger words, the event type, and a role. The electronic device may generate event candidates based on the components of the event. The method by which the electronic device obtains components of an event and generates event candidates is described in greater detail below with reference to FIG. 5.

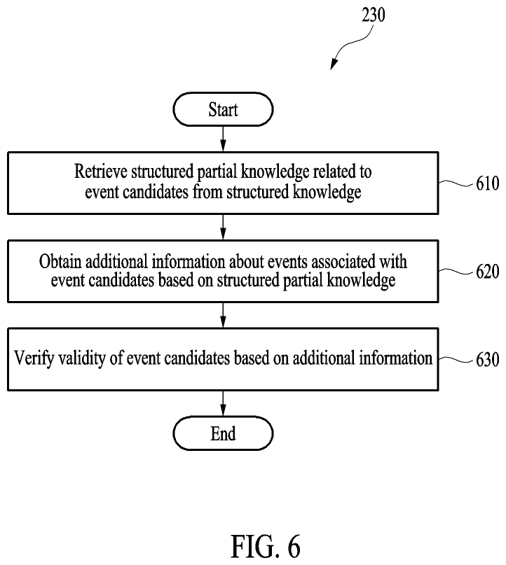

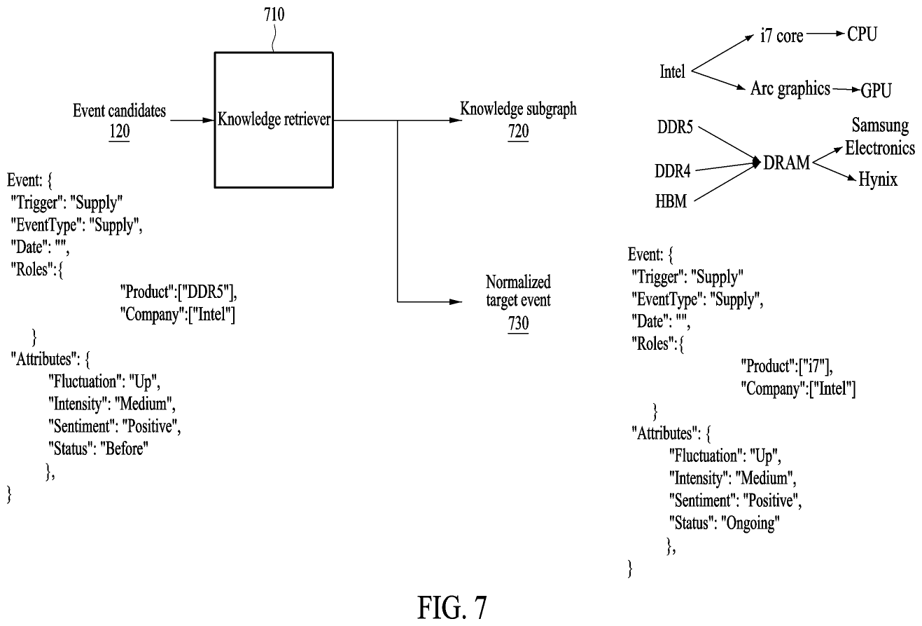

In an example, in operation 230, the electronic device may verify the event candidates generated in operation 220 based on structured knowledge. The structured knowledge may include at least one of a domain knowledge subgraph related to a predetermined domain and an extracted normalized target event. In this case, the structured knowledge may be obtained by the knowledge retriever 130. The electronic device may verify the event candidates by utilizing the structured knowledge of a corresponding domain (e.g., the predetermined domain to which the raw text 101 belongs). The method by which the electronic device verifies event candidates is described in greater detail below with reference to FIGS. 6 and 7.

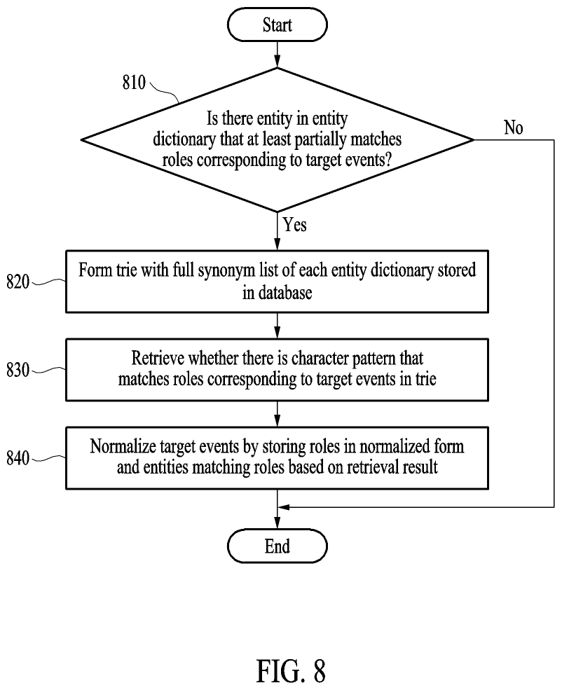

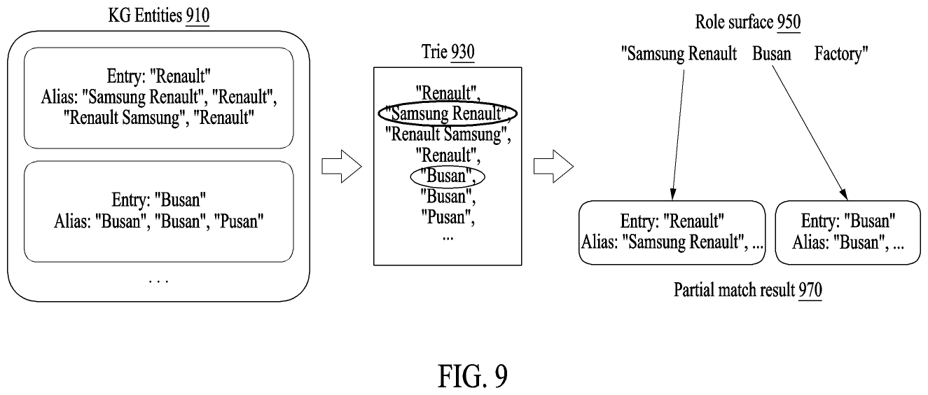

In an example, in operation 240, the electronic device may normalize target events, which are event candidates that are verified in operation 230 among the event candidates using information related to the predetermined domain. The electronic device may normalize the verified target events by matching the verified target events to an entity dictionary including information about the predetermined domain. The electronic device may normalize the target events by normalizing at least one of roles corresponding to the target events and dates corresponding to the target events. The electronic device may normalize the target events based on matching entities, for example, when there are entities in the entity dictionary that at least partially match the roles corresponding to the target events. Alternatively, the electronic device may parse entities matching the dates corresponding to the target events based on a rule when there are entities matching the dates corresponding to the target events in the entity dictionary. The electronic device may normalize the target events by converting the parsed entities into dates and periods based on the date on which the raw text is written. The method by which the electronic device normalizes the verified target events is described in greater detail below with reference to FIGS. 8 and 9.

In an example, in operation 250, the electronic device may extract the normalized target events in operation 240. Additionally, the electronic device may store the normalized target events in a database.

FIG. 3 illustrates an example of raw text and an event according to one or more embodiments. Referring to FIG. 3, in a non-limiting example, an example of raw text 310 and an event 330 are illustrated.

The electronic device (electronic device 1000) may extract an event of a predetermined domain using data of the raw text 310 of the predetermined domain. The raw text 310 may be data such as related news, reports, and meeting minutes. However, examples are not limited thereto. The raw text 310 may be news text such as, for example, “The president of OOO boasted in March that when the United States further blocks the supply of semiconductors, 5G chips may be procured from companies such as Samsung Electronics in Korea or MediaTek in Taiwan.”

The event 330 corresponding to the raw text 310 may have items such as, for example, “trigger words,” “event type,” “date,” “roles,” and “attributes.” The form and/or items of the event 330 may be predefined. The form of the event 330 may vary depending on the intended use of the event 330.

Here, the trigger word corresponding to the raw text 310 is “supply”, the event type is “supply”, there is no date, and the roles may be “product”: [“5G Chip”], “company”: [“Samsung Electronics”, “MediaTek”]. Additionally, attributes corresponding to the raw text 310 may be “Fluctuation: Up”, “Intensity: Medium”, “Sentiment: Positive”, and “Status: Before”. In addition, “Fluctuation” may indicate the direction of change in an event, and “Fluctuation: Up” may indicate that the event progresses in an upward direction (sales increase/decrease, etc.). “Intensity” may indicate the strength (power) of an event, and “Intensity: Medium” may indicate that the event is of average strength (a large increase in sales, etc.). “Sentiment” may indicate whether an event is positive or negative, and “Sentiment: Positive” may indicate that the event is a positive event (a sales event is positive, etc.). “Status” may indicate the point in time of an event, and “Status: Before” may indicate that the event has not yet occurred (e.g. a news article predicts that sales will increase next year->the status is “Before” because the event has not yet occurred).

Trigger words and/or event type may vary from domain to domain. In other words, depending on the domain, the items that trigger words or event type may vary. Additionally, roles and attribute values may vary depending on the changed trigger words and/or event type. As described in greater detail below, a prompt may include questions that fill in the structure of the event described above.

FIG. 4 illustrates an example of input data according to one or more embodiments. Referring to FIG. 4, in a non-limiting example, an example of input data 400 generated based on raw text and a prompt is illustrated.

An electronic (e.g., electronic device 1000) device may use an LLM to extract an event from arbitrary raw text. The electronic device may generate the input data 400 to be input to the LLM by combining the raw text and the prompt. In this case, prompts corresponding to respective items may be predefined so that the respective items of a predefined event may be extracted. The prompts corresponding to respective items may have to be defined so that the LLM may effectively extract the respective items of the event, and the prompts may take various forms.

The electronic device may obtain a response corresponding to the input data 400 by inputting the input data 400 generated using an actual prompt and raw text to the LLM. The electronic device may obtain each component of the event based on the response. Components of the event may be, for example, “role”, “fluctuation”, “intensity”, “sentiment”, and “status” as described above, and “up” in “fluctuation: up” may correspond to an element value of a corresponding component.

In this case, the order of extracting components of the event may be determined depending on whether each component of the event corresponds to the component priority, and a previously extracted item may be included in a prompt for extracting another component. The electronic device may repeat the process described above to ultimately generate event candidates.

However, since a response of an LLM may have uncertainty, there may be risks in directly using event candidates in a real domain. Therefore, in an example, a highly reliable event (i.e., a “normalized target event”) may be finally extracted through an event verification process using additional structured knowledge and a normalization process using predetermined domain information.

FIG. 5 illustrates an example method of event candidate generation with a large language model (LLM) according to one or more embodiments. Referring to FIG. 5, in a non-limiting example, a diagram 500 illustrates a process in which an electronic device (e.g., electronic device 1000) sequentially extracts event candidates by component from a predetermined sentence 501 using an LLM.

The electronic device may sequentially extract components of an event from the predetermined sentence 501 using the LLM. The electronic device may first extract basic component(s) and then extract detailed components based on the extracted components. The predetermined sentence 501 may correspond to a response corresponding to input data. However, examples are not limited thereto.

More particularly, in an example, the electronic device may extract trigger words (515) corresponding to an event from the predetermined sentence 501 by event detection 510. The electronic device may extract an event type 525 corresponding to an event through event classification 520 based on the predetermined sentence 501 and the trigger words 515. In this case, a process of detecting the trigger words 515 and extracting the event type 525 may be performed together.

The electronic device may extract role(s) 535 related to an event by event type by performing event extraction 530 from the predetermined sentence 501 corresponding to a response based on the trigger words 515 and the event type 525. A process of extracting the role(s) 535 related to the event may correspond to a process of extracting a clear entity target, that is, detailed components such as a company, a region, and the like.

The electronic device may extract an attribute value 545 corresponding to an event by performing event attribute classification 540 for the predetermined sentence 501 corresponding to a response based on the trigger words 515, the event type 525 and the role(s) 535. In this way, previously extracted components of an event may be included in the extraction instructions of a subsequent item of an entity.

FIG. 6 illustrates an example method according to one or more embodiments. Referring to FIG. 6, in a non-limiting example, an electronic device (e.g., electronic device 1000) may verify event candidates through operations 610 to 630.

In an example, in operation 610, the electronic device may retrieve structured partial knowledge related to event candidates from structured knowledge. The electronic device may use a knowledge retriever to retrieve a structured partial knowledge graph (KG) related to the event candidates. A method by which the electronic device retrieves a partial KG structured by the knowledge retriever is described in greater detail below with reference to FIG. 7.

In an example, in operation 620, the electronic device may obtain additional information about events associated with the event candidates based on the structured partial knowledge retrieved in operation 610. In this case, the electronic device may extract a corresponding entity and a knowledge subgraph connected to the entity from the structured partial knowledge based on the roles of the event candidates. The knowledge subgraph may correspond to a portion of the KG. The electronic device may use the knowledge subgraph to obtain additional information about the events associated with the event candidates. The electronic device may obtain additional information in a list format about the associated events that overlap in period and target with the event candidates, for example, by using dates corresponding to the event candidates and the roles of the event candidates in the knowledge subgraph.

In an example, in operation 630, the electronic device may verify the validity of the event candidates based on the additional information obtained in operation 620. The electronic device may convert additional information in a list format into a text format. The electronic device may verify the validity of the event candidates by applying the additional information converted into a text format, raw text, and the event candidates to an LLM.

FIG. 7 illustrates an example process of retrieving a structured partial knowledge graph (KG) using a knowledge retriever according to one or more embodiments. Referring to FIG. 7, in a non-limiting example, a process in which an electronic device (e.g., electronic device 1000) obtains a knowledge subgraph 720 and a normalized target event 730 from the event candidate(s) 120 using a knowledge retriever 710 is illustrated.

In an example, the knowledge retriever 170 may retrieve related documents (or related information) for a given input. The knowledge retriever 170 may retrieve related documents by calculating a dot product between a question vector and a document vector to measure similarity using a retrieval model such as dense passage retrieval (DPR) that uses two bidirectional encoder representations from transformers (BERT) models that independently encode questions and documents and selecting the top K documents with the highest relevance based on the similarity calculation result. Alternatively, the knowledge retriever 170 may retrieve related documents by calculating document scores based on word frequency and document length using best matching 25(BM25), a type of probabilistic information retrieval model. When BM25 is used, the more a word appears in a document, the more likely it is that the document is related to that word.

When the event candidate(s) 120 are generated, the electronic device may retrieve the knowledge subgraph 720 associated with components of the event candidate(s) 120, an existing event, and the like using the knowledge retriever 710. The electronic device may verify the reliability of the event candidate(s) 120 by applying the retrieved knowledge subgraph 720 and the existing event as additional information to an LLM.

The electronic device may verify items of the extracted event candidate(s) 120 by utilizing structured knowledge during the verification process. The structured knowledge utilized in this case may include a KG of a related domain, an already extracted event, or the like. The KG is a graph that semantically represents relationships between entities and may include entities (or nodes) and edges (or relations). The KG may be used in various natural language processing (NLP) tasks.

In an example, the electronic device may preferentially retrieve structured knowledge related to the event candidate(s) 120 for verification. The electronic device may basically extract a corresponding entity and the knowledge subgraph 720 connected to the corresponding entity from the KG based on the roles of the event candidate(s) 120. Additionally, the electronic device may obtain a list of related events with overlapping periods and targets by utilizing the dates and roles of the event candidate(s) 120. The electronic device may convert the additional information into a text format and then apply the additional information converted into a text format to the LLM together with the raw text and the event candidate(s) 120 to verify the validity of the event candidate(s) 120.

More particularly, the electronic device may retrieve structured knowledge associated with the event candidate(s) 120 that were previously extracted by the knowledge retriever 710 and verify and/or normalize the event candidate(s) 120 using the retrieved structured knowledge. The electronic device may retrieve structured partial knowledge related to event candidate(s) 120 from the structured knowledge.

The electronic device may obtain additional information about events associated with the event candidate(s) 120 based on the structured partial knowledge. A method by which the electronic device obtains additional information is as follows.

In an example, the electronic device may extract a corresponding entity and the knowledge subgraph 720 connected to the entity from the structured partial knowledge based on the roles of the event candidates 120. The electronic device may obtain additional information about events associated with the event candidate(s) 120 using the knowledge subgraph 720. The electronic device may obtain additional information in a list format about associated events that overlap in period and target with the event candidate(s) 120, for example, by using the dates corresponding to the event candidate(s) 120 and the roles of the event candidate(s) 120 in the knowledge subgraph 720.

The electronic device may verify the validity of the event candidate(s) 120 based on the additional information. The electronic device may, for example, convert the additional information in a list format into a text format and apply the additional information converted into a text format, the raw text, and the event candidate(s) 120 to the LLM to verify the validity of the event candidate(s) 120.

For example, it is assumed that an event candidate such as “[Type: production, Role_product: DDR5, Role_company: Intel, Attribute: Up]”, which indicates that Intel's DDR5 production is improved, is extracted. In this case, the LLM may verify the reliability of the event candidate, that is, the presence of an error in the event candidate, by utilizing the knowledge that “there is no dynamic random access memory (DRAM) among Intel's products” and the knowledge that “DDR5 is a type of DRAM” in the knowledge subgraph 720 related to the event candidate.

This verification process may be gradually improved by training the LLM using error data generated during the event extraction process.

FIG. 8 illustrates an example method according to one or more embodiments. Referring to FIG. 8, in a non-limiting example, an electronic device (e.g., electronic device 1000) may normalize target events through operations 810 to 840.

In an example, in operation 810, the electronic device may determine whether there is an entity in an entity dictionary that at least partially matches (i.e., a partial match) roles corresponding to the target events.

In an example, it is determined that there is a matching entity in operation 810, the electronic device may form a trie with a full synonym list of each entity dictionary stored in a database in operation 820, as illustrated below in FIG. 9.

In an example, in operation 830, the electronic device may retrieve a character pattern that matches the roles corresponding to the target events within the trie that was previously formed in operation 820. That is, if there is a character pattern that matches the roles within the trie, then in operation 820, that character pattern is retrieved.

In an example, in operation 840, the electronic device may normalize the target events by storing normalized roles and entities matching the roles based on the retrieval result in operation 830.

FIG. 9 illustrates an example operation to partially match roles corresponding to target events in a normalization process according to one or more embodiments.

Referring to FIG. 9, in a non-limiting example, a process may include an electronic device (e.g., electronic device 1000) that may form a trie 930 with a full synonym list including entities 910 of each entity dictionary stored in a database and normalizes target events by retrieving whether there is a character pattern that matches roles corresponding to target events in the trie 930.

The electronic device may normalize components respectively corresponding to the event candidates verified through the processes described above. The electronic device may normalize the target events by normalizing at least one of roles corresponding to the target events and dates corresponding to the target events.

In an example, the electronic device may normalize the roles of the target events by utilizing a synonym for an entity name included in the entity dictionary. The electronic device may parse entities matching the dates corresponding to the target events based on a rule when there are entities matching the dates corresponding to the target events in the entity dictionary. The electronic device may normalize the target events by converting the parsed entities into dates and periods based on the date on which the raw text is written. In this case, the electronic device may change date-related expressions in raw text into explicit dates by considering the date of generation of a document, relative expressions, and the like.

In an example, the electronic device may match entities using an entity dictionary, but if only a portion of the entities match the entity dictionary, the entities may be matched using a matching technique based on a trie search. A trie may be a tree-based data structure specialized for storing and retrieving strings. A trie search may be a search based on a trie data structure. In the trie data structure, each node may represent a character of a string and may be stored in a way that a common prefix of strings is shared, enabling efficient searching and insertion. In the trie data structure, each node may store a character of a string and may have a structure that allows a string to be completed by following a path. In addition, in the trie data structure, space efficiency may increase as a common prefix is shared, that is, strings with the same prefix share a common path in a tree, and time complexity may be reduced because searches, insertions, and deletions are performed according to the length of a string. The trie data structure may be useful for processing dictionary-type data or for functions such as string search and auto-completion.

More particularly, in an example, a verified event candidate may be structured into a final event through a process of normalizing the event using information from a related domain. The electronic device may store the final event by normalizing the role and date of an event candidate into a normalized representation. In this process, when there is an entity matching a role in the entity dictionary, the electronic device may normalize the event candidate to the matching entity. In this case, there may be a case in which the representation of the role does not completely match a title in the entity dictionary but only partially matches. Ignoring the case in which only a partial match is found may result in information not being linked, even though the event may be associated with a predetermined entity. Therefore, when the representation of the role only partially matches the title in the entity dictionary, the electronic device may perform matching by utilizing a partial matching rule.

In an example, the electronic device may form the trie 930 with the full synonym list of each entity dictionary stored in the database and retrieve any pattern matching a predetermined event in a role surface 930 based on the trie search. The electronic device may store roles in a normalized form and/or matched entities based on a trie search result. In this case, since the electronic device only matches a character pattern and there is no distinction in role types, homonyms that differ only in type may be stored in both entities.

Additionally, in an example, when there is an entity matching a date in the entity dictionary, the electronic device may parse multiple expressions representing time, such as end of this month, third quarter, and the like, based on a rule and normalize an event candidate to a correct date and period based on the date on which raw text is written. The electronic device may store the final normalized event in a database, which may then be used as structured knowledge for event extraction.

FIG. 10 illustrates an example electronic device according to one or more embodiments. Referring to FIG. 10, in a non-limiting example, an electronic device 1000 may include a memory 1010 and a processor 1030. The electronic device 1000 may further include an LLM 1050. The LLM 1050 may exist in the memory 1030 or may exist separately in the electronic device 1000 as illustrated in FIG. 10. The processor 1010, the memory 1030, and/or the LLM 1050 may be connected to one another via a communication bus.

The electronic device 1000 may include, for example, various computing devices, such as a mobile phone, a smartphone, a tablet, an e-book device, a laptop, a personal computer (PC), a desktop, a workstation, or a server, various wearable devices, such as a smart watch, smart eyeglasses, a head-mounted display (HMD), or smart clothing, various home appliances, such as a smart speaker, a smart television (TV), or a smart refrigerator, and other devices, such as a smart vehicle, a smart kiosk, an Internet of things (IoT) device, a walking assist device (WAD), a drone, or a robot.

The processor 1010 may be one or more and may execute instructions or programs or control the electronic device 1000. The program may generate input data based on, for example, raw text 1001 and a prompt 1003 for a predetermined domain and generate event candidates corresponding to the raw text 1001 by inputting the input data to the LLM 1050. The program may verify the event candidates using structured knowledge, normalize verified target events among the event candidates using information related to the predetermined domain, and extract the normalized target events.

The memory 1030 may include computer-readable instructions. The processor 1010 may be configured to execute computer-readable instructions, such as those stored in the memory 1030, and through execution of the computer-readable instructions, the processor 1010 is configured to perform one or more, or any combination, of the operations and/or methods described herein. The memory 1030 may be a volatile or nonvolatile memory.

The processor 1010 may be configured to execute programs or applications to configure the processor 1010 to control the electronic device 1000 to perform one or more or all operations and/or methods involving the resolution of a deadlock state and resuming a task, and may include any one or a combination of two or more of, for example, a graphics processing unit (GPU), a neural processing unit (NPU), a tensor processing unit (TPU), and the like. Additionally, in some examples, the processor 1010 may include a central processing unit (CPU).

In an example, the processor 1010 may generate the input data based on the raw text 1001 and the prompt 1003 for the predetermined domain. The processor 1010 may generate the event candidates corresponding to the raw text 1001 by inputting the input data to the LLM 1050. The processor 1010 may verify the event candidates using structured knowledge expressed as a KG 1005 and normalize verified target events among the event candidates using information related to the predetermined domain. The processor 1010 may extract the normalized target events.

The processor 1010 may perform the operations described above with reference to FIGS. 1 to 9 as at least some of the instructions stored in the memory 1030 are executed by one or more processors 1010.

The electronic devices, apparatuses, neural networks, memories, processors, large language model (LLM) 110, knowledge retriever 130, electronic device 1000, processor 1010, memory 1030, LLM 1050, described herein and disclosed herein described with respect to FIGS. 1-10 are implemented by or representative of hardware components. As described above, or in addition to the descriptions above, examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-10 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and/or any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A processor-implemented method, the method comprising:
generating input data based on raw text and a prompt for a predetermined domain;
generating, through a large language model (LLM), event candidates corresponding to the raw text;
verifying the event candidates based on structured knowledge as target events;
normalizing the target events using information related to the predetermined domain; and
extracting the normalized target events.

2. The method of claim 1, wherein the prompt is predefined for respective items of a predefined event to extract the respective items, and
wherein the predefined event comprises a syntax comprising any one or any combination of trigger words corresponding to the predetermined domain, an event type corresponding to the event, a role related to the event, and an attribute value corresponding to the event.

3. The method of claim 1, wherein the generating of the event candidates comprises:
obtaining, through the LLM, components of a detected event from the raw text; and
generating the event candidates based on the components of the detected event.

4. The method of claim 3, wherein the obtaining of the components of the event comprises:
extracting, through the LLM, trigger words corresponding to the detected event and an event type corresponding to the event;
extracting, through the LLM, a role related to the detected event by the event type based on the trigger words and the event type; and
extracting, through the LLM, an attribute value corresponding to the detected event, based on the trigger words, the event type, and the role.

5. The method of claim 3, wherein the obtaining of the components of the event comprises:
determining an extraction order of the components of the event based on a priority of the components of the detected event; and
obtaining the components of the event according to the extraction order.

6. The method of claim 1, wherein the structured knowledge comprises one or more of a knowledge subgraph of a domain related to the predetermined domain and the extracted normalized target event.

7. The method of claim 1, wherein the verifying of the event candidates comprises:
retrieving structured partial knowledge related to the event candidates from the structured knowledge;
obtaining additional information about an event associated with the event candidates based on the structured partial knowledge; and
verifying a validity of the event candidates based on the additional information.

8. The method of claim 7, wherein the obtaining of the additional information comprises:
extracting, from the structured partial knowledge, a corresponding entity and a knowledge subgraph connected to the corresponding entity, based on roles of the event candidates; and
obtaining additional information about an event associated with the event candidates using the knowledge subgraph.

9. The method of claim 8, wherein the obtaining of the additional information about the event associated with the event candidates using the knowledge subgraph comprises:
obtaining, using dates corresponding to the event candidates and the roles of the event candidates in the knowledge subgraph, the additional information in a list format about associated events that overlap in period and target with the event candidates.

10. The method of claim 7, wherein the verifying of the validity of the event candidates based on the additional information comprises:
converting the additional information in a list format into a text format; and
verifying the validity of the event candidates by applying the additional information converted into a text format, the raw text, and the event candidates to the LLM.

11. The method of claim 1, wherein the normalizing of the target events comprises:
normalizing the target events by matching the verified target events to an entity dictionary comprising information about the predetermined domain.

12. The method of claim 1, wherein the normalizing of the target events comprises:
normalizing the target events by standardizing one or more of roles corresponding to the target events and dates corresponding to the target events.

13. The method of claim 12, wherein the normalizing of the target events comprises:
normalizing the target events based on the matching entity responsive to a partially matching entity partially matching the roles corresponding to the target events, being in an entity dictionary.

14. The method of claim 13, wherein the normalizing of the target events based on the partially matching entity comprises:
forming a trie with a full synonym list of each entity dictionary of a plurality of dictionaries;
retrieving a character pattern from within the trie, the retrieved character pattern matching roles of the target events; and
normalizing the target events by storing roles in a normalized form and entities matching the roles based on the retrieved character pattern.

15. The method of claim 12, wherein the normalizing of the target events comprises:
in response to dates of an entity matching target event dates corresponding to the target events in an entity dictionary, parsing the matching entity based on a rule; and
normalizing the target events by converting the parsed entity into a date and a period based on a date on which the raw text is written.

16. The method of claim 1, further comprising:
storing the normalized target events in a database.

17. A non-transitory, computer-readable storage medium storing instructions that, when executed by the one or more processors, configure the one or more processors to:
generate input data based on raw text and a prompt for a predetermined domain;
generate, through a large language model (LLM), event candidates corresponding to the raw text;
verify the event candidates based on structured knowledge as target events;
normalize the target events using information related to the predetermined domain; and
extract the normalized target events.

18. An electronic device, comprising:
processors configured to execute instructions; and
a memory storing the instructions, wherein execution of the instructions configures the processors to: generate input data based on raw text and a prompt for a predetermined domain;
generate, through a large language model (LLM), event candidates corresponding to the raw text;
verify the event candidates based on structured knowledge as target events;
normalize the target events using information related to the predetermined domain; and
extract the normalized target events.

19. The electronic device of claim 18, wherein the prompt is predefined for respective items of a predefined event to extract the respective items, and
wherein the event comprises a syntax comprising any one or any combination of trigger words corresponding to the predetermined domain, an event type corresponding to the predefined event, a role related to the predefined event, and an attribute value corresponding to the predefined event.

20. The electronic device of claim 18, wherein the processor is configured to:
extract, through the LLM, trigger words corresponding to a detected event from the raw type and an event type corresponding to the detected event;
extract, through the LLM, a role related to the detected event by the event type based on the trigger words and the event type;
extract, through the LLM, an attribute value corresponding to the detected event, based on the trigger words, the event type, and the role; and
generate the event candidates based on components of the detected event comprising any one or any combination of the trigger words, the event type, the role, and the attribute value.
